(19) United States
(12) Patent Application Publication
(10) Pub. No.: US 2018/0085392 A1
(43) Pub. Date: Mar. 29, 2018

(54) CYCLODEXTRIN COMPOUNDS FOR THE PREVENTION AND TREATMENT OF AGING

(71) Applicant: SENS RESEARCH FOUNDATION, INC., Mountain View, CA (US)

(72) Inventors: Jason Thomas GASPAR, Kingwood, TX (US); Jacques MATHIEU, Houston, TX (US); Pedro J.J. ALVAREZ, Houston, TX (US); Aubrey DE GREY, Los Gatos, CA (US)

(21) Appl. No.: 15/567,121

(22) PCT Filed: Apr. 15, 2016
(86) PCT No.: PCT/US16/27996

§ 371 Date: Oct. 17, 2017

Related U.S. Application Data
(60) Provisional application No. 62/149,278, filed on Apr. 17, 2015.

(51) Int. Cl. A61K 31/724; A61K 47/69; A61K 9/08; A61K 9/14

(52) U.S. Cl. CPC A61K 31/724; A61K 47/6951; A61K 2300/00; A61K 9/146; A61K 9/08

(57) ABSTRACT

Cyclodextrin compound therapeutics to reduce age-related non-bisretinoid lipofuscin in a patient having a buildup of same, said method comprising administering 2-hydroxypropyl-β-cyclodextrin, or its derivatives, or β-cyclodextrin or its derivatives, or α-cyclodextrin or its derivatives, or γ-cyclodextrin or its derivatives in an amount effective to reduce non-bisretinoid lipofuscin levels. The methods include treating and/or preventing aging of the skin, muscle and other tissues and organs, which may result in increased life-span or health-span.

### PRIOR RELATED APPLICATIONS

This Application claims the benefit of U.S. Provisional Patent Application Ser. No. 62/149,278, filed Apr. 17, 2015, which is hereby incorporated by reference in its entirety.

### FIELD OF THE DISCLOSURE

The disclosure generally relates to therapeutic methods and compositions utilizing cyclodextrins and derivatives thereof. In exemplary embodiments, these compositions and methods are suitable for removal of disease-associated products that accumulate with age and are associated with age-related disease, including lipofuscins.

### BACKGROUND OF THE DISCLOSURE

Cyclodextrins (“CDs”) (sometimes called cycloamyloses) are a family of compounds made up of sugar molecules bound together in a ring (cyclic oligosaccharides). Cyclodextrins can be produced from starch by means of enzymatic conversion. They are used in food, pharmaceutical, drug delivery, and chemical industries, as well as agriculture and environmental engineering.

Cyclodextrins are composed of 5 or more α-D-glucopyranoside units linked 1->4, as in amylose (a fragment of starch). The 5-membered macrocycle is not natural. The largest well-characterized cyclodextrin contains 32 1,4-anhydroglucopyranoside units, while as a poorly characterized mixture, at least 150-membered cyclic oligosaccharides are also known. Typical cyclodextrins contain a number of glucose monomers ranging from six to eight units in a ring, creating a cone shape;

α (alpha)-cyclodextrin: 6-membered sugar ring molecule

β (beta)-cyclodextrin: 7-membered sugar ring molecule

γ (gamma)-cyclodextrin: 8-membered sugar ring molecule

For example, see FIG. 1A for the general structure of these three cyclodextrins. Due to the chair conformation of the glucopyranose units, the cyclodextrins are shaped like a truncated cone rather than perfect cylinders (FIG. 1B).

Since cyclodextrins are hydrophobic inside and hydrophilic outside, they can form complexes with hydrophobic compounds. Thus, cyclodextrins can enhance the solubility and bioavailability of such hydrophobic compounds. This property is of high interest for pharmaceutical as well as dietary supplement applications in which hydrophobic compounds shall be delivered. Alpha-, beta-, and gamma-cyclodextrin are all generally recognized as safe (“GRAS”) as a food additive by the FDA.

Cyclodextrins can be produced by the treatment of starches with enzymes. Commonly, cyclodextrin glycosyltransferase (“CGTase”) is employed along with α-amylase. First, starch can be liquefied either by heat treatment or by using α-amylase, then CGTase can be added for the enzymatic conversion. CGTases can synthesize all forms of cyclodextrins, thus the product of the conversion usually results in a mixture of the three main types of cyclic molecules in ratios that can be dependent on the enzyme used: each CGTase has its own characteristic α:β:γ synthesis ratio. CGT can be obtained from Bacillus macerans, B. circulans, or related strains of Bacillus.

Purification of the three types of cyclodextrins can take advantage of the different water solubility of the molecules: β-cyclodextrin, which solubilizes in water at a ratio of 18.5 g/L (16.3 mM) at 25° C., can be retrieved through crystallization, while the more soluble α- and γ-cyclodextrins (solubility of 145 and 232 g/l in water, respectively) are usually purified by means of expensive and time consuming chromatography techniques.

As an alternative, a “complexing agent” can be added during the enzymatic conversion step: such agents (usually organic solvents like toluene, acetone, or ethanol) can form a complex with the desired cyclodextrin that then subsequently precipitates. The complex formation can drive the conversion of starch towards the synthesis of the precipitated cyclodextrin, thus enriching its content in the final mixture of products. Some manufacturers use dedicated enzymes that can produce alpha-, beta- or gamma-cyclodextrin specifically. Specific production of cyclodextrin is employed in the food industry, as alpha- and gamma-cyclodextrin can be consumed without a daily intake limit.

Cyclodextrins can form host-guest complexes with hydrophobic molecules given the unique nature imparted by their truncated cone structure (see FIG. 1B). As a result, cyclodextrins have found a number of applications in a wide range of fields.

Cyclodextrins can solubilize hydrophobic drugs in pharmaceutical applications and crosslink to form polymers used for drug delivery. One example is sugammadex, a modified γ-cyclodextrin that can reverse neuromuscular blockade by binding the drug rocuronium.

Other than the above-mentioned pharmaceutical applications, cyclodextrins can be employed in environmental protection: these molecules can immobilize toxic compounds, such as trichloroethane or heavy metals, inside of their rings, or these molecules can form complexes with stable substances, like trichlorfon (an organophosphorus insecticide) or sewage sludge, thereby enhancing their decomposition.

Both β-cyclodextrin and methyl-β-cyclodextrin (“MβCD”) can remove cholesterol from cultured cells, and the methylated form, MβCD, was found to be more efficient at this removal process than β-cyclodextrin. The water-soluble MβCD can form soluble inclusion complexes with cholesterol, thereby enhancing its solubility in aqueous solution. MβCD can be employed for the preparation of cholesterol-free products: the bulky and hydrophobic cholesterol molecule can become lodged inside cyclodextrin rings that are then subsequently removed. MβCD can also be employed (e.g., in research) to disrupt lipid rafts, which is thought to result from its ability to remove cholesterol from membranes.

Toxicity studies have demonstrated that when administered orally cyclodextrins are generally non-toxic due to lack of absorption from the gastrointestinal tract. Indeed, only negligible amounts of hydrophilic cyclodextrins and drug/cyclodextrin complexes were able to permeate lipophilic membranes such as gastrointestinal mucosa and skin.

Certain lipophilic cyclodextrin derivatives, such as the methylated cyclodextrins, are surface active and they are to some extent (˜10%) absorbed from the gastrointestinal tract; consequently limited amounts of these lipophilic cyclodextrin derivatives have been included in oral formulations, potentially limiting their suitability for parenteral formulations. Due to toxicological considerations, beta-cyclodextrin may be less suitable for parenteral formulations. However, extensive toxicological studies have been completed for HPβCD as well as for sulfobutylether beta-cyclodextrin, both of which can be found in marketed parenteral formulations at relatively high concentrations.

In addition to oral use, HPβCD has been used as an intrathecal drug vehicle. Beta cyclodextrins have also been used in pulmonary, nasal, sublingual injectable, ophthalmic, and dermal drug delivery. Thus, it appears that the beta cyclodextrins are well tolerated.

One cyclodextrin, 2 hydroxypropyl beta-cyclodextrin, has been used as an inactive ingredient in certain household products such as FEBREZE® and as a component of a formulation of SPORANOX® (itraconazole, an anti-fungal capsule). Initiation of a Phase I trial to evaluate use of HPβCD in a treatment regimen for Niemann-Pick disease, type C has been reported. This is a rare and fatal lysosomal storage disease that can cause progressive deterioration of the nervous system and dementia. In addition, HPβCD has been used as anti-epileptic drug.

However, to date, such cyclodextrins have not been used for anti-aging treatments, and such a use, described herein, represent a new treatment paradigm for conditions associated with aging.

### SUMMARY OF THE DISCLOSURE

The present disclosure provides for the use of cyclodextrins to remove non-bisretinoid lipofuscins from cells. Such methods allow restoration of a more youthful phenotype in aged cells and tissues, as these lipofuscins generally increase in concentration with age and are believed to interfere with cellular functions. Anti-aging therapeutics containing one or more cyclodextrins, such as 2-hydroxypropyl-beta-cyclodextrin (“HPβCD”, “2-HPβCD”, or “HβCD”), are provided.

Treatment with HPβCD can reduce non-bisretinoid lipofuscin from human skin cells. This finding is significant because non-bisretinoid lipofuscin accumulation occurs in tissues throughout the body as humans age, including skin, heart, and brain tissue as well as skeletal muscle. Non-bisretinoid lipofuscin accumulation can be detrimental to cell health and functioning. Therefore, treatment with HPβCD may be used to rejuvenate cells to a healthier condition, postpone disease onset, and lessen symptom severity for age-related disorders.

Lipofuscin is a brown-yellow, autofluorescent polymeric material that can accumulate within postmitotic cells during aging. The mitochondrial-lysosomal axis theory of aging posits that oxidizing conditions within the cell, prevalent as a result of mitochondrial metabolic processes, can lead to Fenton-type crosslinking reactions. These crosslinking reactions can create recalcitrant, nondegradable autofluorescent material (i.e. lipofuscin) that eventually accumulates within cells.

This accumulation of nondegradable autofluorescent material is the basis for the “garbage catastrophe” theory of aging. The continued accumulation of lipofuscin is theorized to result in a decline in overall cell health and homeostasis. Lipofuscin has been reported to accumulate within lysosomes in cells, though its accumulation inside cells may not be restricted to lysosomes. Lipofuscin accumulation within the lysosomes can inhibit proper lysosome and proteasome function. Likewise, lipofuscin accumulation in other organelles or other cellular locations may impair cellular functions. As aging progresses, lipofuscin can progressively accumulate in cells. In some organisms, such as crustaceans, lipofuscin can accumulate in a linear fashion, thereby enabling the age of the organism to be determined by measuring the total lipofuscin content of said organism.

The identification of drugs that can reduce or can clear lipofuscin would be beneficial for the treatment of aging and geriatrics. Lipofuscin accumulation often occurs in tissues with high oxidative demand such as the brain and heart. However, significant lipofuscin accumulation has been noted in most post-mitotic and senescent cells of the body, including skin and muscle cells. A treatment that removes or slows the accumulation of lipofuscin could be used for a variety of aging treatments. Removing or slowing lipofuscin accumulation may rejuvenate skin cells, muscle cells, cardiomyocytes, and neurons to a younger and/or healthier state.

Flow cytometry experiments demonstrated that a 5-day treatment with HPβCD reduced non-bisretinoid lipofuscin by 31.4% in aged human skin cells (p<0.00001) (see Table 3 below). This finding indicated that HPβCD may be developed into an anti-aging treatment. Additional microscopy results and RT-qPCR results as well as data obtained from cell assays and cell-free assays also support the above results.

We hypothesize that HPβCD can prevent lipid oxidation and can mitigate the detrimental effects of oxidized lipids by increasing subcellular flux, thereby preventing the accumulation of lipid deposits and facilitating appropriate metabolism. For example, incorporation of oxidized lipids, including but not limited to the cholesterol byproduct 7-ketocholesterol, into the lysosomal membrane can increase membrane permeability, can impair membrane protein function through lipid raft disruption, and may result in phospholipidosis. Notably, incorporation of 7-ketocholesterol into membranes also can result in increased production of reactive oxygen species, thereby potentially leading to lipofuscin accumulation. Without intending to be limited by theory, it is believed that therapies that decrease the concentration of oxidized lipids may augment proper lysosome function and thereby may slow or decrease the accumulation of lipofuscin. For example, treatment using HPβCD may result in decreased concentration of oxidized lipids such as 7-ketocholesterol.

Cyclodextrins, such as HPβCD, or other derivatives of β-cyclodextrin, such as methyl and ethyl derivatives, as well as a-cyclodextrins, y-cyclodextrins and their derivatives, can be used to formulate drug therapeutics for age-related disorders and other disorders related to the aging or premature aging of cells and cell structures, including but not limited to cardiovascular and cerebrovascular diseases. Exemplary age-related disorders or conditions include cardiovascular, neurodegenerative, eye diseases, inflammatory diseases and conditions, and age-related diseases and conditions. Exemplary cardiovascular diseases and conditions include arteriosclerosis, coronary heart disease, arrhythmia, heart failure, hypertension, orthostatic hypotension, myocardial infarction, angina pectoris, heart failure, atherosclerosis, stroke, renal artery disease or stenosis, peripheral vascular disease, chronic obstructive pulmonary disease (“COPD”), chronic renal diseases with renal failure, and heart disease. Exemplary neurodegenerative diseases include Parkinson's disease and Alzheimer's disease. Exemplary eye diseases and conditions include macular degeneration, Stargardt disease, cataracts, diabetic retinopathy, and glaucoma. Exemplary inflammatory diseases and conditions include arthritis, such as rheumatoid arthritis. Additional exemplary age-associated diseases and disorders include ulcers, osteopetrosis, progeria, weakness, hearing loss, and type-2 diabetes. Any of the above mentioned cyclodextrin compounds, when used as therapeutics, may improve overall health of cells by slowing or reversing the cellular aging process, or by causing cells to exhibit one or more phenotypes of younger cells. It is to be understood that “age-related” refers to diseases frequently associated with aging, however, a given patient need not be of advance age, but rather the subject methods and compositions may he used regardless of the patient's age

Exemplary cyclodextrins may also be used to “rejuvenate” cells and/or tissues containing non-bisretinoid lipofuscin, especially cells and tissues known to accumulate high levels of non-bisretinoid lipofuscin. Such cells and tissues include without limitation thereto cardiomyocytes, brain cells and/or tissues, skeletal muscle, smooth muscle, retinal pigment epithelium, and other tissue types containing non-bisretinoid lipofuscin.

Thus, the invention includes the following embodiments in any combination of one or more thereof:
A method of treating geriatric aging of the human or animal body, tissue or organ is provided, comprising administering a cyclodextrin compound:

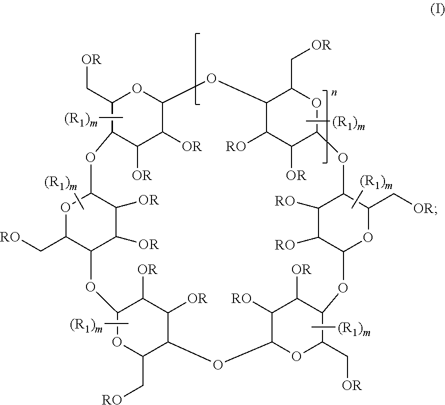

or a pharmaceutically acceptable salt, ester, solvate, or hydrate thereof, wherein, each R is independently H, alkyl, alkenyl, alkynyl, cycloalkyl, heterocycloalkyl, aryl, or heteroaryl, each of which is optionally substituted; or —C(O)OR^{B}, —OC(O)R^{B}, —C(O)R^{B}, or —C(O)NR^{A}R^{B};
each R₁ is independently H, alkyl, alkenyl, alkynyl, cycloalkyl, aryl, heteroaryl, halogen, hydroxy, amino, —CN, —CF₃, —N₃, —NO₂, —OR^{B}, —SR^{B}, —SOR^{B}, —SO₂R^{B}, —N(R^{B})S(O₂), —R^{B}, —N(R^{B})S(O₂)NR^{A}R^{B}, —NR^{A}R^{B}, —C(O)OR^{B}, —OC(O)R^{B}, —C(O)R^{B}, —C(O)NR^{A}R^{B}, or —N(R^{B})C(O)R^{B}; each of which is optionally substituted;
each R^{A} is independently hydrogen, alkyl, alkenyl, alkynyl, cycloalkyl, heterocycloalkyl, aryl, or heteroaryl, each of which is optionally substituted;
each R^{B} is independently hydrogen, alkyl, alkenyl, alkynyl, cycloalkyl, heterocycloalkyl, aryl, or heteroaryl, each of which is optionally substituted;
n is 1, 2, 3, 4, 5, 6, 7, 8, 9, or 10; and
each m is independently 0, 1, 2, 3, 4, or 5.
   In certain embodiments, each R is independently H, optionally substituted alkyl, —C(O)OR^{B}, —OC(O)R^{B}, —C(O)R^{B}, or —C(O)NR^{A}R^{B}
   In a further embodiment, each R is independently H, methyl, ethyl, propyl, butyl, pentyl, hexyl, heptyl, or octyl; wherein each is straight chain or branched.
   In other embodiments, n is 1, 2, or 3.
   In another embodiment, the cyclodextrin is 2-hydroxypropyl-β-cyclodextrin, or its derivatives, or β-cyclodextrin or its derivatives, or α-cyclodextrin or its derivatives, or γ-cyclodextrin or its derivatives or a pharmaceutically acceptable salt, ester, solvate, or hydrate thereof.
   A method of treating geriatric aging of the body, comprising parentally or orally administering a composition comprising 2-hydroxypropyl-β-cyclodextrin, or its derivatives, or β-cyclodextrin or its derivatives, or α-cyclodextrin or its derivatives, or γ-cyclodextrin or its derivatives solubilized in parentally acceptable solution.
   A method of treating geriatric aging of the body, comprising 2-hydroxypropyl-β-cyclodextrin, or its derivatives, or β-cyclodextrin or its derivatives, or α-cyclodextrin or its derivatives, or γ-cyclodextrin or its derivatives topically, parenterally, or orally administering a composition comprising or a derivative thereof solubilized in parenterally acceptable solution.
   A method of treating geriatric aging of the body, comprising administering a composition comprising 2-hydroxypropyl-β-cyclodextrin, or its derivatives, or β-cyclodextrin or its derivatives, or α-cyclodextrin or its derivatives, or γ-cyclodextrin or its derivatives, solubilized in parenterally acceptable solution.
   A method of reducing non-bisretinoid lipofuscin in a patient having a buildup of same, said method comprising administering 2-hydroxypropyl-β-cyclodextrin, or its derivatives, or β-cyclodextrin or its derivatives, or α-cyclodextrin or its derivatives, or γ-cyclodextrin or its derivatives, to a patient in an amount effective to reduce non-bisretinoid lipofuscin levels in said patient.

### BRIEF DESCRIPTION OF THE DRAWINGS

FIG. 1A. provides a representation of the chemical structures of alpha, beta and gamma cyclodextrins.

FIG. 1B provides a representation of toroidal view of a beta-cyclodextrin.

FIG. 1C provides a representation of 2-Hydroxypropyl-β-cyclodextrin.

FIG. 2 provides representative confocal photomicrographs of fibroblasts. The far left photomicrograph presents a healthy young fibroblast, the middle photomicrograph presents an aged fibroblast, and the far right photomicrograph presents an aged fibroblast that was treated with HPβCD.

FIGS. 3A-3B provide analysis of cells treated normal LDL (“nLDL”), oxidized LDL (“oxLDL”), or 7-ketocholesterol-LDL (“7KC-LDL”), with or without treatment with HPβCD (“HβCD”). FIG. 3A. Traces of absorbance versus the ratio of acridine orange (“AO”) red to AO green fluorescence. FIG. 3B. Average flow cytometry signal intensities as a percent of control (normal LDL-treated cells). These results indicate that cyclodextrin treatment attenuated lysosomal destabilization/permeability caused by 7-ketocholesterol.

FIG. 4 provides lipofuscin levels obtained by flow cytometry measurement of fluorescence, resulting from a 5-day treatment of aged human skin cells with HPβCD (“2-HPCD”). Lipofuscin levels were elevated in aged human skin cells, and which were decreased after the HPβCD treatment.

FIG. 5 provides representative confocal photomicrographs wherein the accumulation of lipofuscin in fibroblasts that were either treated with HPβCD (top row of photomicrographs) or were not treated with HPβCD (bottom row of photomicrographs) was imaged over the course of 8 days. Representative photomicrographs from day 4, day 6, and day 8 are provided for each the treated and untreated fibroblasts (top and bottom rows of photomicrographs, respectively).

FIG. 6 provides representative data quantifying the autofluorescence of lipofuscin loaded cells (“PC”) either without cyclodextrin treatment (“PC only”) or treated with HPβCD (“PC+cyclodextrin”). Fluorescence indicates relative levels of lipofuscin present within the treated or untreated PC cells after 10 days (left column) or 12 days (right column). Between days 10 and 12, fluorescence decreased by 13% in the PC only cells, and by 23% in the PC cells treated with HPβCD.

FIG. 7A-B provide representative photomicrographs of the autofluorescence of native lipofuscin from either untreated fibroblasts (FIG. 7A, labeled untreated) or fibroblasts treated with HPβCD (FIG. 7B, labeled treated). Autofluorescence was visibly decreased in the cells treated with HPβCD. The same cells under visible light (60× magnification) are shown in FIG. 7C and FIG. 7D, respectively. Cells are shown after 16 days of cell population maintenance.

FIG. 8 provides results of a CCK-8 assay that was performed on cells over the course of a 10 day period. Results from day 4, day 7, and day 10 are displayed along with bar graphs corresponding to the quantification of cell viability as measured by the CCK-8 assay as described infra. A circle around a well indicates a blank control well as described infra.

FIG. 9 provides representative data from a RT-PCR analysis of major lysosome genes ULK1, TFEB, and HMOX from healthy cells treated with HPβCD (“NC w/CD”), lipofuscin loaded cells (“PC”), and lipofuscin loaded cells treated with HPβCD (“PC w/CD”) following 10 days of cell population maintenance.

FIG. 10A-D provides representative photomicrographs of filipin-stained cell populations either untreated (FIG. 10A) or HPβCD-treated for 3.5 hours (FIG. 10C) before confluency, as well as filipin-stained cell populations untreated (FIG. 10B) or HPβCD-treated for 3.5 hours (FIG. 10D) following four days of cell maintenance.

FIG. 11A-D provides representative photomicrographs of filipin-stained untreated healthy cells (FIG. 11A, “NC”), healthy cells treated with HPβCD (FIG. 11B, “NC w/CD”), untreated cells loaded with lipofuscin (FIG. 11C, “PC”), and cells loaded with lipofuscin and treated with HPβCD (FIG. 11D, “PC w/CD”) at 100× magnification following four days of cell maintenance. The staining shows that cholesterol levels were increased by treatment.

FIG. 12A-D provides representative photomicrographs of untreated healthy cells (FIG. 12A, “NC”), healthy cells treated with HPβCD (FIG. 12B, “NC w/CD”), untreated cells loaded with lipofuscin (FIG. 12C, “PC”), and cells loaded with lipofuscin and treated with HPβCD (FIG. 12D, “PC w/CD”) at 200× magnification following nine days of cell maintenance.

FIG. 13A-D provides representative photomicrographs of untreated healthy cells (“NC”, FIG. 13A), untreated cells loaded with lipofuscin (“PC”, FIG. 13B), healthy cells treated with HPPCD (“NC w/CD”, FIG. 13C), and cells loaded with lipofuscin and treated with HPβCD (“PC w/CD”, FIG. 13D) after exposure to 45 μM of MSDH for 42 hours.

FIG. 14A-D provides representative photomicrographs of untreated healthy cells (“NC”, FIG. 14A), untreated cells loaded with lipofuscin (“PC”, FIG. 14B), healthy cells treated with HPβCD (“NC w/CD”, FIG. 14C), and cells loaded with lipofuscin and treated with HPβCD (“PC w/CD”, FIG. 14D) after the populations were exposed to a higher level of MSDH for a longer duration (55 μM for 42 hours).

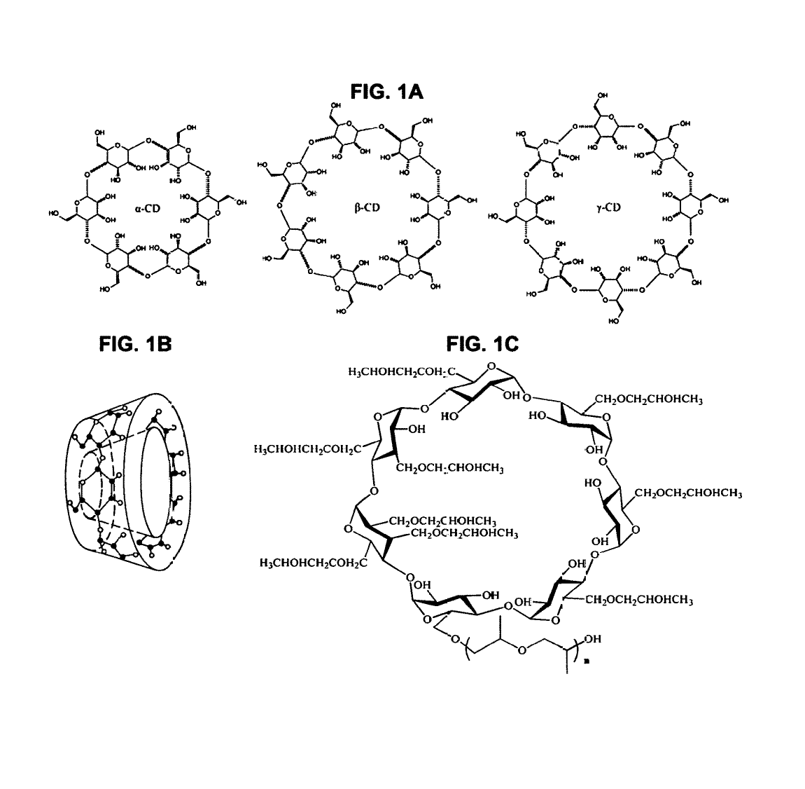

### DETAILED DESCRIPTION

The use of the term “or” in the claims is used to mean “and/or” unless explicitly indicated to refer to alternatives only, or if the alternatives are mutually exclusive.

The use of the word “a” or “an” when used in conjunction with the term “comprising” in the claims or the specification means one or more than one, unless the context dictates otherwise.

The abbreviations “HPβCD”, “2-HPβCD”, and “HβCD”, are all understood to abbreviate 2-hydroxypropyl-β-cyclodextrin and are used interchangeably herein.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention, such as instructions for use, buffers, flavorants, excipients and other inactive ingredients, and the like.

“Derivative,” as used herein, includes but is not limited to, amide, ether, ester, amino, carboxyl, acetyl, and/or alcohol derivatives of a given compound.

“About” where used means especially +/−10%, +/−5% or +/−3% (referring to the given numeric value), if not indicated otherwise. In each of the invention embodiments, “about” can be deleted.

As used herein, “bisretinoid” shall mean a compound which has two retinoid molecules linked together and includes A2E, A2E isomers and compounds of the all-trans-retinal dimer series, i.e., dimers of trans isomers of the retinal compound.

Non-bisretinoid lipofuscin refers to any lipofuscin other than bisretinoid lipofuscin, most especially lipofuscin that decreases in concentration in cells treated with a cyclodextrin. Lipofuscin accumulates with aging in cells and tissues including liver, kidney, heart muscle, retina, adrenals, nerve cells, and ganglion cells, in both intra- and extra-cellular locations. Levels of lipofuscin can be measured using autofluorescence methods as disclosed herein, based on coloration, and other assays known in the art. Lipofuscin may also be detected based on functional consequences of lipofuscin accumulation, such as pigmentation, impairment lysosome function, etc.

“Liver spot” (interchangeably, “age spot”, “solar lentigo”, “Lentigo senilis”, “old age spot,” or “senile freckle”) refers to skin blemishes associated with aging, typically light brown to red or black. Liver spots typically derive their color from the accumulation of lipofuscin, including within skin cells and within extracellular lipofuscin bodies.

“Administering” a compound can be effected or performed using any of the various methods and delivery systems known to those skilled in the art. The administering can be performed, for example, intravenously, orally, nasally, via the cerebrospinal fluid, via implant, transmucosally, transdermally, intramuscularly, intraocularly, parenterally, topically and subcutaneously. The following delivery systems, which employ a number of routinely used pharmaceutically acceptable carriers, are only representative of the many embodiments envisioned for administering compositions according to the instant methods.

Injectable drug delivery systems include solutions, suspensions, gels, microspheres and polymeric injectables, and can comprise excipients such as solubility-altering compounds (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating compounds (e.g., starch polymers and cellulosic materials) and lubricating compounds (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid) .

Dermal delivery systems (e.g., topical delivery systems) include, for example, aqueous and nonaqueous gels, creams, multiple emulsions, microemulsions, liposomes, ointments, aqueous and non-aqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrrolidone). In one embodiment, the pharmaceutically acceptable carrier is a liposome or a transdermal enhancer.

Solutions, suspensions and powders for reconstitutable delivery systems include vehicles such as suspending compounds (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine) , preservatives and antioxidants (e.g., parabens, vitamins E and C, and ascorbic acid), anti-caking compounds, coating compounds, and chelating compounds (e.g. , EDTA) .

In the practice of the method, administration may comprise daily, weekly, monthly or hourly administration, the precise frequency being subject to various variables such as age and condition of the subject, amount to be administered, half-life of the compound in the subject, area of the subject to which administration is desired and the like. In exemplary embodiments, 200-600 mg HP-β-CD is administered by injection (e.g., intravenously, subcutaneously, on intraperitoneally) twice a week for 8-20 weeks, e.g., for the treatment of conditions related to 7KC and/or lipofuscin accumulation. In further exemplary embodiments, a prophylactic regimen may comprise administration of a low dose, such as less than 300, less than 200, less than 100, or less HP-β-CD per dose. Said dosage may be administered by injection (e.g., intravenously, subcutaneously, on intraperitoneally), e.g., once or twice per week. Said dosage may be administered for a prolonged duration, such as repeated administration over the course of at least three months, at least six months, at least one year, at least two years, at least three years, at least five years, or longer.

As used herein, “lipofuscin-associated disorder” shall mean disorders associated with an increased accumulation of lipofuscin in cells. These disorders may include, but are not limited to Age-related Macular Degeneration (AMD), Stargardt disease, Best disease, lipofuscinoses, e.g., neuronal ceroid lipofuscinoses, also known as Batten disease, Alzheimer's disease, Parkinson's disease, amyotrophic lateral sclerosis, certain lysosomal diseases, acromegaly, denervation atrophy, lipid myopathy, chronic obstructive pulmonary disease, centronuclear myopathy or any disease which is correlated with an increased accumulation of lipofuscin.

The disclosure provides novel treatments for age-related conditions comprising administering one or more cyclodextrins. Also provided are therapeutic compositions comprising cyclodextrin which may be adapted for use in treatment of age-related conditions. In exemplary embodiments, the cyclodextrin may comprise 2-hydroxypropyl-β-cyclodextrin or a derivative thereof, or β-cyclodextrin or a derivative thereof, or α-cyclodextrin or a derivative thereof, or γ-cyclodextrin or a derivative thereof.

In exemplary embodiments, the cyclodextrin is administered in order to prevent or treat the development of an age-related condition.

As used herein, “treatment” refers to an approach for obtaining beneficial or desired clinical results. Treating a subject or patient refers to any indication of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation.

As used herein, an “effective dosage” or “effective amount” of drug, compound, or pharmaceutical composition is an amount sufficient to effect beneficial or desired results. For prophylactic use, beneficial or desired results include results such as eliminating or reducing the risk, lessening the severity, or delaying the outset of the disease, including biochemical, histological, and/or behavioral symptoms of the disease, its complications and intermediate pathological phenotypes presenting during development of the disease.

“Prophylaxis” or “prevention” or “delaying the onset” refers to slowing the progression of or development of a disease, condition, symptom, or disorder (the terms disease, condition, and disorder are used interchangeably throughout the application). Delaying the onset may be determined based upon a reduction in severity or slowing of progression relative to the absence of treatment. Reduction in severity includes reducing drugs and/or therapies generally used for the condition by, for example, reducing the need for, amount of, and/or exposure to drugs or therapies. Reduction in severity also includes reducing the duration, and/or frequency of the particular condition, symptom, or disorder. Prevention may be determined using diagnostic tests or biomarkers, e.g., using biomarkers associated with aging or an age-related condition. In the case of lipofuscin accumulation, prevention may refer to maintenance of the amount of lipofuscin at or below a level determined prior to or at the onset of treatment, or relative to the condition in the absence of a treatment (e.g., relative to controls, which may be based upon historical outcome data).

For example, a cyclodextrin may be administered to a subject who does not show the signs or symptoms of a disease or condition associated with aging. Said therapeutic regimen is expected to delay or prevent the onset of said disease or condition. A subject may be administered a cyclodextrin starting prior to the typical age of onset of symptoms, e.g., a subject less than 40, less than 35, less than 30, less than 25, or less than 20 years of age. A subject may show symptoms of the early stages of a disease associated with aging. For example, a subject may show pre-hypertension, metabolic syndrome, and/or fatty streaks indicative of pre-cardiovascular disease, e.g., detectable by virtual angiogram.

Said treatment may be administered at a low dose. Without intent to be limited by theory, it is believed that a low dose may be particularly effective for prophylaxis, such that progression of an age-associated disease or condition may be slowed or delayed. For example, the dosage may be less than one half, one fifth, or one tenth of a dosage associated with acute toxicity. As one example, the dosage may be selected to be one half, one fifth, or one tenth of a dosage associated with hearing loss. Said treatment may be administered over a long period of time, e.g., repeated administration of a cyclodextrin over a course of months or years. In exemplary embodiments, cyclodextrin may be repeatedly administered over the course of at least three months, at least six months, at least one year, at least two years, at least three years, at least five years, or longer. Adjustment to the dosage may be made based upon the size, weight, gender, health condition, and other characteristics of the subject. Additionally, the dosage may be adjusted based upon the characteristics of a particular cyclodextrin, such as its solubility, potency, and any associated toxicity or side-effects.

In exemplary embodiments, the cyclodextrin is administered in order to prevent or treat the accumulation of pigmentation in the skin, e.g., liver spots.

Exemplary embodiments provide topical formulations, e.g., for application to the skin, comprising a cyclodextrin and a carrier or excipient. Exemplary topical formulations may be suitable for treating the upper skin layers. Exemplary topical formulations may be suitable for transdermal administration of cyclodextrin to local tissue underlying the skin or into the blood for systemic distribution. Examples of formulations that may be used for topical or transdermal administration are provided in U.S. Pub. Nos. 2011/0028460, 2014/0010902, 2016/0058693 each of which is hereby incorporated by reference in its entirety.

Exemplary embodiments provide for ophthalmic formulation, e.g., eye drops or an injection solution. An injection solution may be suitable for injection into the eye, e.g., Tenon's capsule or ocular fundus. For example, an ophthalmic formulation may comprises a solubilizer such as polyethylene glycol 400, glycerin, etc.; a stabilizer such as EDTA etc.; a buffering agent such as boric acid etc.; a pH controlling agent such as hydrochloric acid, sodium hydroxide, phosphoric acid, citric acid, sodium bicarbonate etc. An ophthalmic formulation may include another active agent, examples of which include anti-inflammatory agents such as epsilon-aminocaproic acid, dipotassium glycyrrhizinate, dicrofenac sodium and pranoprofen; vasoconstrictors such as phenylephrine hydrochloride, naphazoline hydrochloride and tetrahydrozoline hydrochloride; anti-allergic drugs such as sodium cromoglicate and ketotifen fumarate; antihistamine drugs such as chlorpheniramine maleate and diphenhydramine hydrochloride, antiseptics such as benzalkonium chloride, paraoxybenzoate ester, sorbic acid and chlorobutanol. Opthalmic formulations may include surfactants such as polyoxyethylene, hydrogenated castor oil and polyoxyethylene sorbitan monooleate; vitamins such as pyridoxine hydrochloride, riboflavin phosphate, cyanocobalamin, panthenol, tocophenol acetate, and flavin adenine dinucleotide sodium; amino acids such as sodium chondroitin sulfate, potassium L-aspartate, and aminoethylsulfonic acid; and inorganic salts such as sodium chloride and potassium chloride.

Exemplary cyclodextrin derivatives are shown in Table 1:

TABLE 1
CD derivatives

| Cyclodextrin | Abb. | R | n |
|---|---|---|---|
| β-cyclodextrin | β-CD | H | 5 |
| Carboxymethyl-β-cyclodextrin | CM-β-CD | CH2C02H or H | 5 |
| Carboxymethyl-ethyl-β-cyclodextrin | CME-β-CD | CH2C02H, | 5 |
| | | CH2CH3 or H | |
| Diethyl-β-cyclodextrin | DE-β-CD | CH2CH3 or H | 5 |
| Dimethyl β cyclodextrin | DM-β-CD | CH3 or H | 5 |
| Glucosyl-β-cyclodextrin | G1-β-CD | Glucosyl or H | 5 |
| Hydroxybutenyl-β-cyclodextrin | HBU-β-CD | CH2CH(CHCH2)OH | 5 |
| | | or H | |
| Hydroxyethyl-β-cyclodextrin | HE-β-CD | CH2CH20H or H | 5 |
| Hydroxypropyl-β-cyclodextrin | HP-β-CD | CH2CHOHCH3 or H | 5 |
| Hydroxypropyl-γ-cyclodextrin | HP-γ-CD | CH2CHOHCH3 or H | 6 |
| Maltosyl-β-cyclodextrin | G2-β-CD | Maltosyl or H | 5 |
| Methyl-β-cyclodextrin | M-β-CD | CH3 or H | 5 |
| Random methyl-β-cyclodextrin | RM-β-CD | CH3 or H | 5 |
| Sulfobutylether-β-cyclodextrin | SBE-β-CD | (CH2)4S03Na or H | 5 |

a Derivatives may have differing degrees of substitution on the 2, 3, and 6 positions

The oral absorption of certain cyclodextrins and their derivatives following oral administration is found in Table 2.

TABLE 2
oral absorption of cyclodextrins

| | | Dose | Absorption |
|---|---|---|---|
| Cyclodextrin | Species | (mg/kg) | (% dose) |
| a-CD | rat | 200 | 1 |
| β-CD | rat | 600 | 0.6 |
| γ-CD | rat | 1,000 | 0.02 |
| G-β-CD | rat | 500 | 0.3 |
| HP-β-CD | rat | 40 | 3 |
| SBE-β-CD | rat | 60 | 1.64 |

In exemplary embodiments, a cyclodextrin composition may be suitable for topical application. Exemplary compositions may include one or more of moisturizers, depigmenting or propigmenting agents, antimicrobial agents, anti-pollution agents or free-radical scavengers, agents for neutralizing monocyclic or polycyclic aromatic compounds; heavy-metal neutralizing agents, NO-synthase inhibitors, agents for stimulating the synthesis of dermal or epidermal macromolecules and/or for preventing their degradation, agents for stimulating the proliferation of fibroblasts or keratinocytes and/or keratinocyte differentiation, dermo-decontracting agents, tensioning agents, calmatives, agents acting on the capillary circulation, and agents acting on the energy metabolism of cells. See, e.g., U.S. Pub. No. 2005/0164991, which is hereby incorporated by reference in its entirety.

Exemplary antimicrobial agents include 2,4,4′-trichloro-2′-hydroxydiphenyl ether (or triclosan), 3,4,4′-trichlorobanilide, phenoxyethanol, phenoxypropanol, phenoxyisopropanol, hexamidine isethionate, metronidazole and its salts, miconazole and its salts, itraconazole, terconazole, econazole, ketoconazole, saperconazole, fluconazole, clotrimazole, butoconazole, oxiconazole, sulfaconazole, sulconazole, terbinafine, ciclopirox, ciclopiroxolamine, undecylenic acid and its salts, benzoyl peroxide, 3-hydroxybenzoic acid, 4-hydroxybenzoic acid, phytic acid, N-acetyl-L-cysteine acid, lipoic acid, azelaic acid and its salts, arachidonic acid, resorcinol, 2,4,4′-trichloro-2′-hydroxydiphenyl ether, 3,4,4′-trichlorocarbanilide, octopirox, octoxyglycerine, octanoylglycine, caprylyl glycol, 10-hydroxy-2-decanoic acid, dichlorophenyl imidazole dioxolane and its derivatives, described in patent WO 93/18743, farnesol and phytosphingosines, and mixtures thereof The preferred antibacterial agents are triclosan, phenoxyethanol, octoxyglycerine, octanoylglycine, 10-hydroxy-2-decanoic acid, caprylyl glycol, farnesol and azelaic acid. By way of example, the antimicrobial agent may be used in the composition according to the invention in an amount representing from 0.1% to 20% and preferably from 0.1% to 10% relative to the total weight of the composition.

The active compounds described herein may be formulated for administration in a pharmaceutical carrier in accordance with known techniques. See, e.g., Remington, The Science And Practice of Pharmacy (LWW; Twenty-First edition, 2005), herein incorporated by reference. In the manufacture of a composition according to several embodiments of the invention, the active compound (including the physiologically acceptable salts thereof) may be admixed with, inter alia, an acceptable carrier. The carrier is acceptable in the sense of being compatible with any other ingredients in the composition and must not be deleterious to the patient. The carrier may be a solid or a liquid, or both, and may be formulated with the compound as a unit-dose formulation, for example, a tablet, which may contain from 0.01% or 0.5% to 95% or 99% by weight of the active compound. One or more active compounds may be incorporated in the compositions of the invention, which may be prepared by any of the well-known techniques of pharmacy comprising admixing the components, optionally including one or more accessory ingredients. Carriers which may be used include petroleum jelly, lanoline, polyethylene glycols, alcohols, transdermal enhancers, and combinations of two or more thereof In some embodiments, the topical composition is partially or fully incorporated in delivery vehicles such as microspheres or nanoparticles, or are encapsulated (e.g., in liposomes). In some embodiments, a topical composition is pre-impregnated in/on a support structure (e.g., tape, patch, bandage, etc.).

In addition to one or more active compounds, the topical compositions disclosed herein may comprise other additives, such as pH-adjusting additives. In some embodiments, pH-adjusting agents include acids, such as citric acid or lactic acid, bases or buffers, such as sodium lactate, sodium acetate, sodium phosphate, sodium citrate, sodium borate, or sodium gluconate.

The compositions may contain antimicrobial preservatives in some embodiments. In several embodiments, antimicrobial preservatives include, but are not limited to, methylparaben, propylparaben, benzyl alcohol, ethylhexylglycerin, potassium sorbate, phenoxyethanol, EDTA, grapefruit seed extract, tea tree oil, sodium benzoate, dehydroacetic acid, and combinations thereof. In some embodiments, anti-fungal preservatives are used alone or in combination with anti-bacterial preservatives. In one embodiment, the topical compositions are paraben-free. In one embodiment, an antimicrobial preservative is used when the formulation is placed in a vial designed for multi-dose use. In some embodiments, the compositions disclosed herein comprise anti-biofilm anti-microbial agents such as lactoferrin, xylitol, farnesol gallium, dispersin B, EDTA, and furanone compounds, or combinations thereof. The compositions according to some embodiments may be lyophilized.

In several embodiments, the topical compositions disclosed herein comprise agents that improve barrier structure and function, including, but not limited to, polyols, ceramides, sterols, plant extracts, peptides. Specific ingredients include one or combinations of the following as examples: xylitylglucoside, anhydroxylitol, xylitol, ceramide 2, ceramide 3, hydroxypropyl bispalmitamide MEA, glycine soya sterols, niacinamide, or salts/acids thereof.

In several embodiments, the topical compositions disclosed herein comprise agents that reduce glycation, including, but not limited to, plant extracts, vitamins, peptides. Specific ingredients include one or combinations of the following as examples: Centella Asiatica, vitamin B-6, and L-carnosine, and extracts or salts/acids thereof.

In several embodiments, the topical compositions disclosed herein comprise antioxidants. Antioxidants may include, but are not limited to, superoxide dismutase, catalase, glutathione peroxidase, glutathione, ascorbic acid, and tocopherol.

The following examples are provided in order to illustrate the invention. These following examples are intended to be illustrative only, and not to limit the scope of the appended claims. Additionally, throughout the examples, the terms “CD” or “cyclodextrin” and the like are used to refer to HPβCD, unless indicated otherwise.

### EXAMPLES

### Example 1

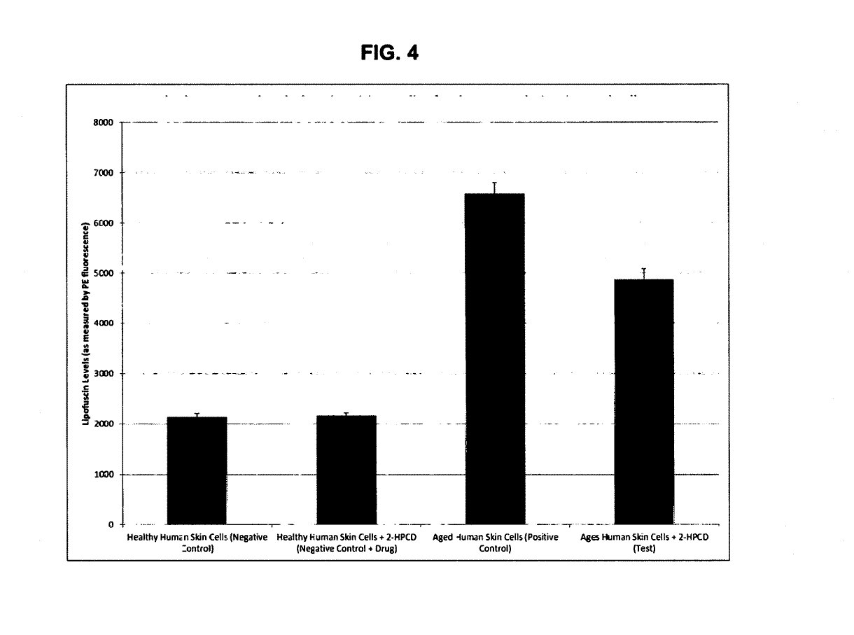

Flow cytometry results (FIG. 4 and Table 3) demonstrated that a 5-day treatment with HPβCD reduced non-bisretinoid lipofuscin by 31.4% in aged human skin cells (p<0.00001). This finding indicated that HPβCD may be used as a treatment for age-associated conditions, particularly, conditions mediated directly or indirectly by non-bisretinoid lipofuscin. Table 3 and FIG. 4 presents data obtained from the treatment of human fibroblast cells with 10 rng/ml 2HPβCD for five days.

The values of Table 3 represent total non-bisretinoid lipofuscin content measured in fluorescent units per cell using a BD FACSCANTO™ II flow cytometer (BD Biosciences, San Jose, Calif.). Measurements were made with a 488 nm blue argon laser with a 530/30 nm FITC band pass filter. The total cell population size for each individual sample measurement was n>15,000 cells.

TABLE 3
Reduction in non-bisretinoid lipofuscin

| | Sample | Sample | Sample | Sample | | Standard |
|---|---|---|---|---|---|---|
| | 1 | 2 | 3 | 4 | Average | Error |
| Human Skin Cells | 2630 | 2660 | 2830 | 2734 | 2714 | 89 |
| (Negative Control) | | | | | | |
| Human Skin Cells + 2-HPCD | 2552 | 2564 | 2638 | 2647 | 2600 | 49 |
| (Negative Control + Drug) | | | | | | |
| Aged Human Skin Cells | 8628 | 8933 | 9308 | 8875 | 8936 | 281 |
| (Positive Control) | | | | | | |
| Aged Human Skin Cells + | 6278 | 6453 | 5982 | 5809 | 6131 | 289 |
| 2-HPCD (Test) | | | | | | |

Additional microscopy results, cell assays, and cell-free assays confirmed these results and provided further supporting information for the possible mechanism of action.

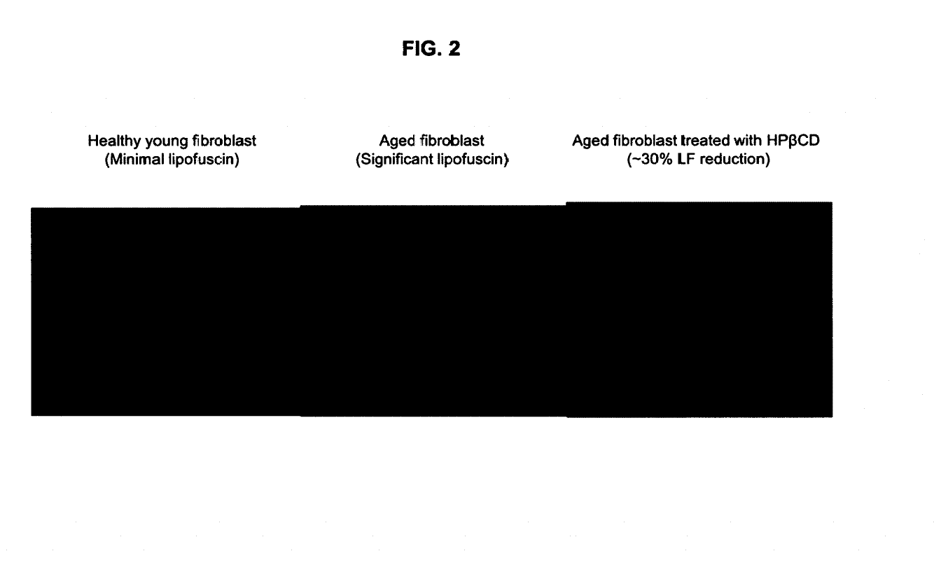

FIG. 2 provides representative confocal microscopy pictures, wherein Healthy Young Fibroblast (far left photomicrograph) showed minimal Lipofuscin (“LF”). Aged Fibroblasts in contrast showed significant lipofuscin (middle microphotograph), and aged fibroblasts treated with beta cyclodextrins showed a ˜30% LF reduction (far right microphotograph).

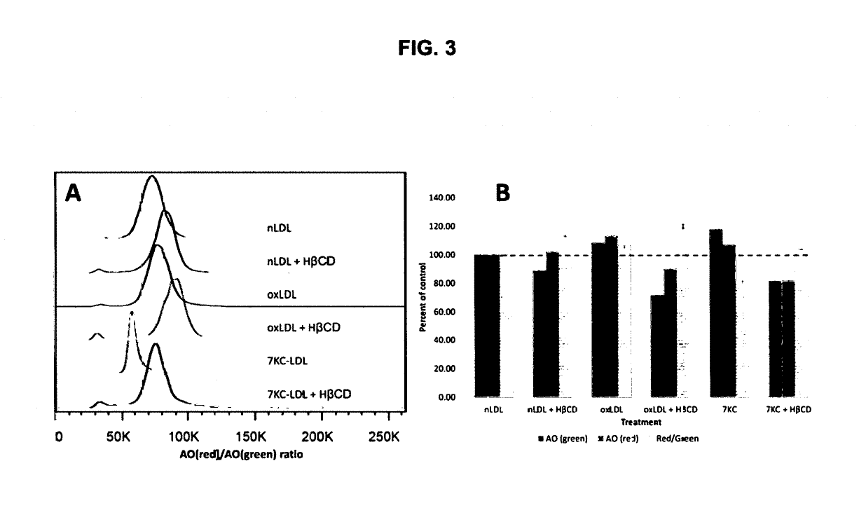

FIG. 3A-B shows data indicating that HPβCD attenuated the lysosomal destabilization/permeability caused by 7-ketocholesterol. These data indicate that HPβCD helps maintain lysosome function under conditions of stress.

To prepare FIG. 3A-B, THP-1 cells were exposed to different LDL treatments over 7 days and then assessed for lysosomal membrane permeabilization using an acridine orange uptake assay.

In FIG. 3A: Acridine orange was excited at 488 nm and emissions recorded at 633 and 520 nm. Higher signal intensities at 520 nm and lower intensities at 633 nm were indicative of lysosomal instability. 7KC-LDL induced lysosomal membrane permeabilization that was attenuated by HβCD that was added to samples on day 6.

In FIG. 3B: Flow cytometry signal are presented as intensities as a percent of control (normal LDL-treated). HβCD treatment improved the 633 nm/520 nm ratio in all cases. Although both the 633 nm and 520 nm signal intensities decreased after HβCD amendment for oxLDL and 7KC-LDL, the decrease in the 520 nm signal was of greater magnitude, thereby indicating increased lysosomal stability.

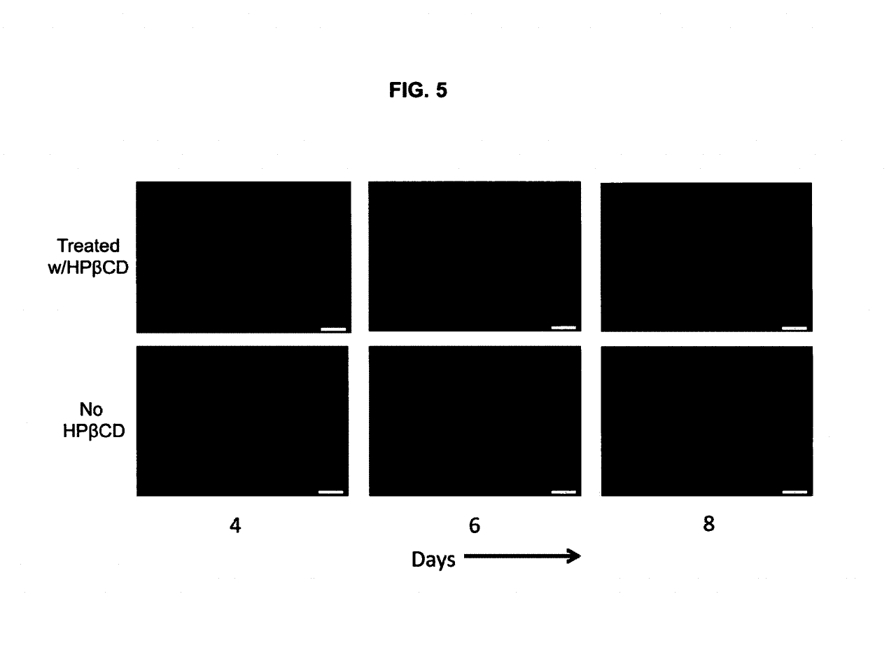

FIG. 5 provides representative confocal photomicrographs wherein the accumulation of lipofuscin in fibroblasts that were either treated with HPβCD or were not treated with HPβCD was fluorescently imaged over the course of 8 days. After 8 days, the cells treated with HPβCD displayed lower lipopigment accumulation (i.e. fluorescence) as compared to fibroblasts that had not been treated with HPβCD.

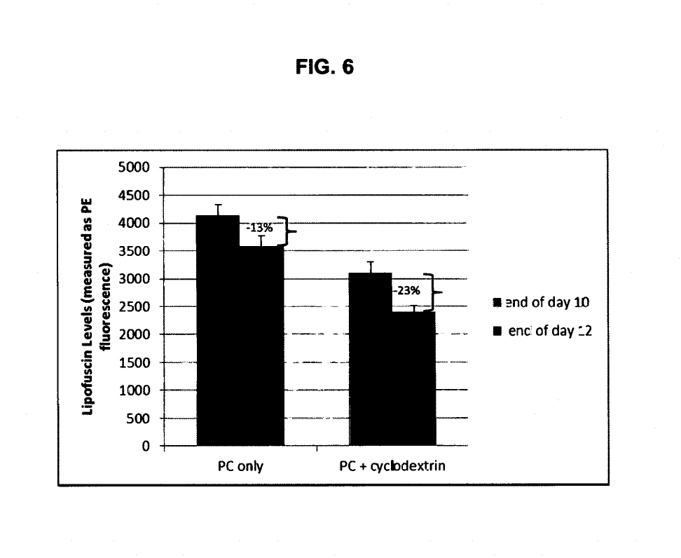

FIG. 6 provides representative quantified data of the autofluorescence of lipofuscin loaded cells (PC cells) (human skin fibroblasts, Coriell Cell Repositories, Catalog #GM00498) either untreated or treated with CD in order to track the removal of lipofuscin already present within the PC cells. To perform the assay, PC cells were allowed to grow for 5 days, and the PC cells were treated such that lipofuscin was produced only from day 1 through day 12. After 5 days, one population of cells (“treated population”, PC+cyclodextrin in FIG. 6) was treated with cyclodextrin from day 5 to day 10, whereas a second population of cells (“untreated population”, PC only in FIG. 6) remained untreated from day 5 to day 10. On day 10, lipofuscin levels were measured for both cell populations. Following this measurement, the untreated population was placed in media only for 48 hours whereas the treated population was placed in media containing cyclodextrin for 48 hours. 48 hours following this transfer of the cell populations to their respective media, the lipofuscin levels of both the untreated and treated populations were again measured. A 23% decrease in autofluorescence from day 10 to day 12 was observed for the treated population, whereas a 13% decrease was observed for the untreated population. The decrease in autofluorescence observed in the treated population over the course of 48 hours (from day 10 to day 12) indicated that the cyclodextrin treatment was responsible for removing pre-existing lipofuscin that was present the PC cells. The p-value comparing the percentage differences between the treated and untreated populations was 0.05. Note that the 13% decrease from day 10 to day 12 in the untreated population was interpreted to be likely due to cell division, as the cell cycle of the untreated population was not arrested, and therefore the percentage of autofluorescence would decrease as the untreated population of cells divided.

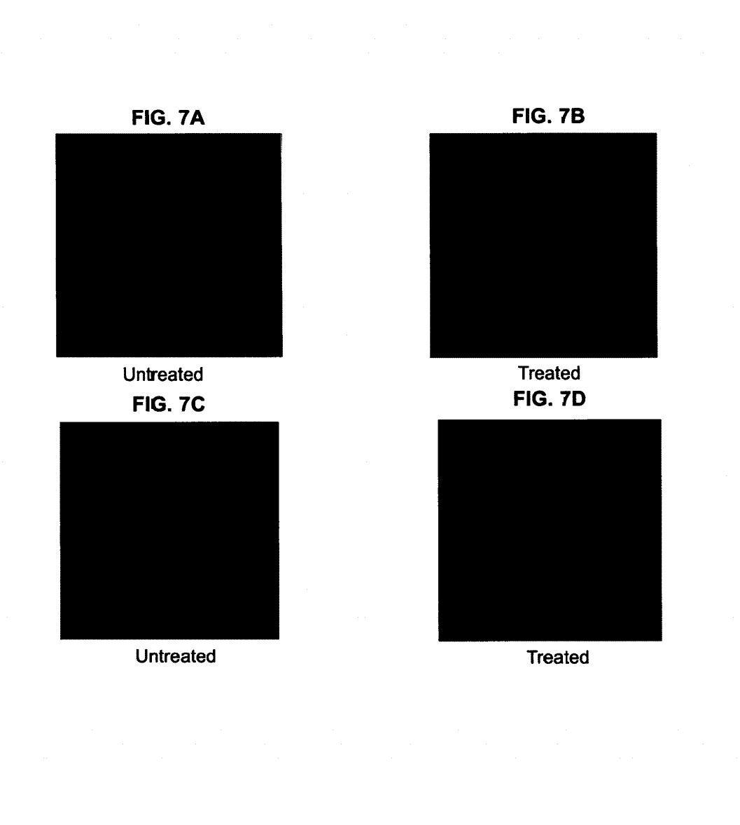

FIG. 7A-B provides representative photomicrographs that demonstrated the ability of cyclodextrin treatment to decrease the native lipofuscin concentration in fibroblasts. Healthy fibroblasts were passaged (˜P25) and grown to confluency in media. The cells in FIG. 7A (“untreated”) were maintained for 16 days; likewise, the cells in FIG. 7B (“treated”) were maintained for 16 days, however, cyclodextrin was added to the treated cells during days 1-8. Following the 16 day period of cell maintenance, lipofuscin levels were observed using fluorescence imaging as lipofuscin appears as a bright red autofluorescent material during fluorescence-based imaging. The cyclodextrin-treated cells of FIG. 7B displayed significantly reduced levels of lipofuscin autofluorescence as compared to the untreated cells of FIG. 7A, suggesting that cyclodextrin was capable of inducing removal of native lipofuscin from the fibroblasts. In order to ascertain whether or not the untreated or treated fibroblasts appeared healthy after the 16 day period of maintenance, both the untreated and treated fibroblasts were imaged with 60× magnification using light microscopy. The representative photomicrographs of FIG. 7C and FIG. 7D showed that both the untreated and treated fibroblasts appeared to be healthy following the 16 day period.

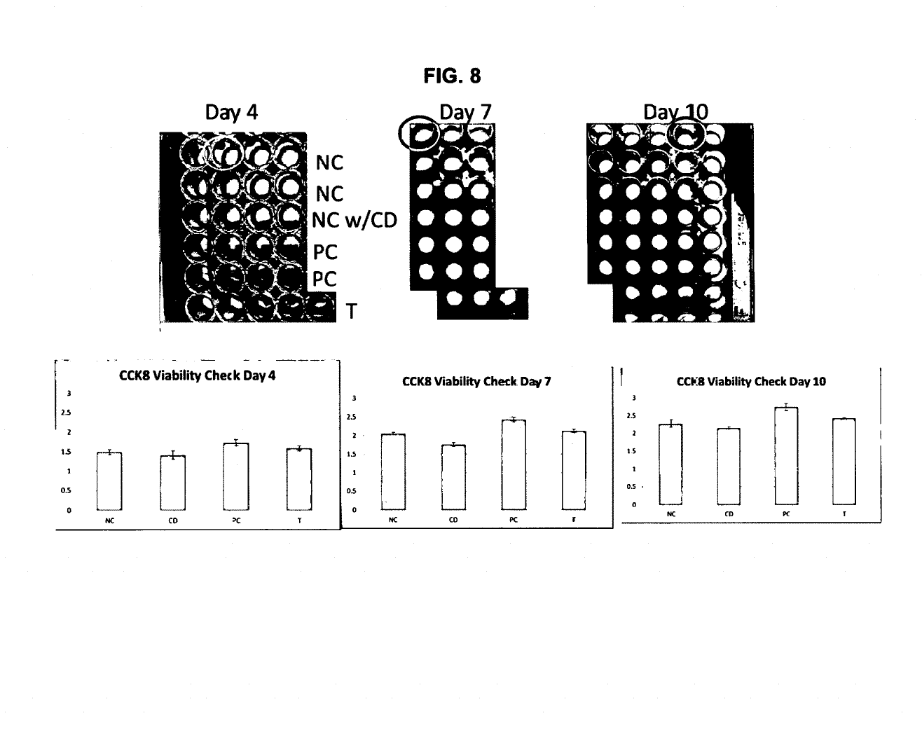

In order to determine whether cells were still viable when treated with cyclodextrin, a CCK-8 assay was performed on cells (human skin fibroblasts, Coriell Cell Repositories, Catalog #GM00498) over the course of a 10 days period (FIG. 8). On each day 4, day 7, and day 10, the viability of the cells was ascertained through a CCK-8 assay whereby colorimetric change was indicative of change in the cell viability status. The cell populations tested on day 4, day 7, and day 10 were healthy negative control cells (NC), healthy cells treated with cyclodextrin (NC+CD), lipofuscin loaded cells (PC), and lipofuscin loaded cells treated with cyclodextrin (T). For all three days tested (days 4, 7, and 10) no visible colorimetric difference between healthy negative control cells (NC), healthy cells treated with cyclodextrin (NC+CD), lipofuscin loaded cells (PC), and lipofuscin loaded cells treated with cyclodextrin (T) was observed. FIG. 8 also presents blanks that were used as controls (the blanks appear circled in red in FIG. 8). The results of the CCK-8 assay indicated that the lipofuscin loaded cells and lipofuscin loaded cells treated with cyclodextrin appeared as viable as were the healthy untreated cells.

### Example 2

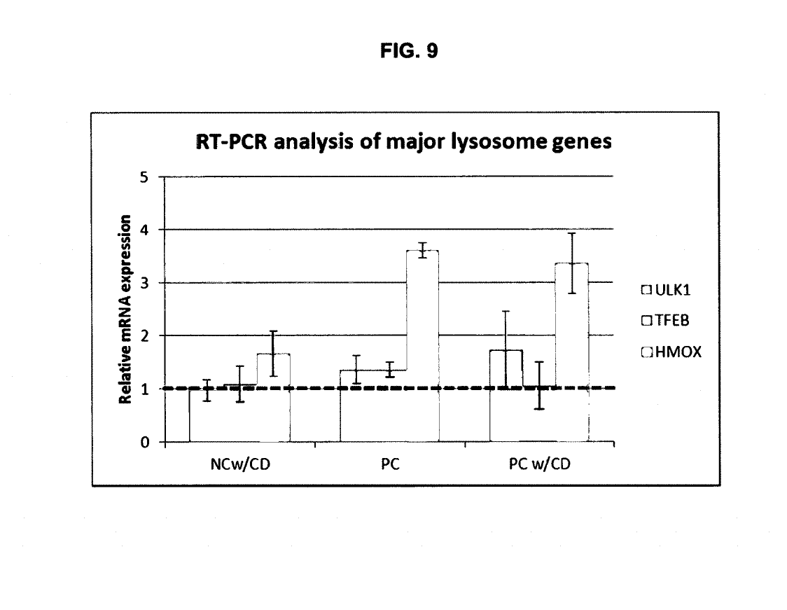

To address the mechanisms by which cyclodextrin treatment can remove lipofuscin from cells, a RT-PCR analysis of major lysosome genes was performed (FIG. 9). Healthy cells treated with cyclodextrin (“NC w/CD”), lipofuscin loaded cells (“PC”), and lipofuscin loaded cells treated with cyclodextrin (“PC w/CD”) were maintained for 10 days. Following the 10 day period, RT-PCR was performed on each cell population in order to ascertain the regulation status of the major lysosome genes ULK1, TFEB, and HMOX. In this experiment, HMOX was used as a positive control gene as it was upregulated by the addition of iron, an addition that only occurred in the PC and PC w/CD cell populations. The results of FIG. 9 demonstrated that the cell populations treated with cyclodextrin displayed no upregulation of either ULK1 or TFEB at day 10 as compared to the PC cell population that was not treated with cyclodextrin.

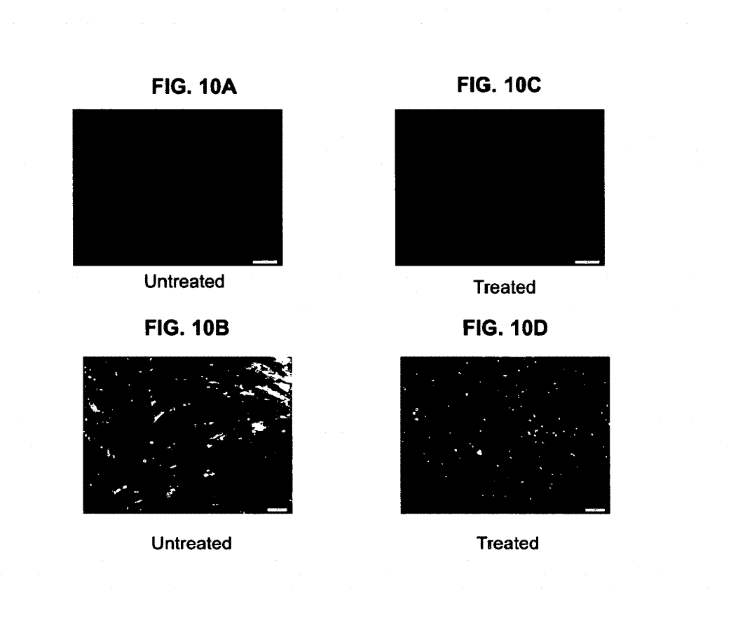

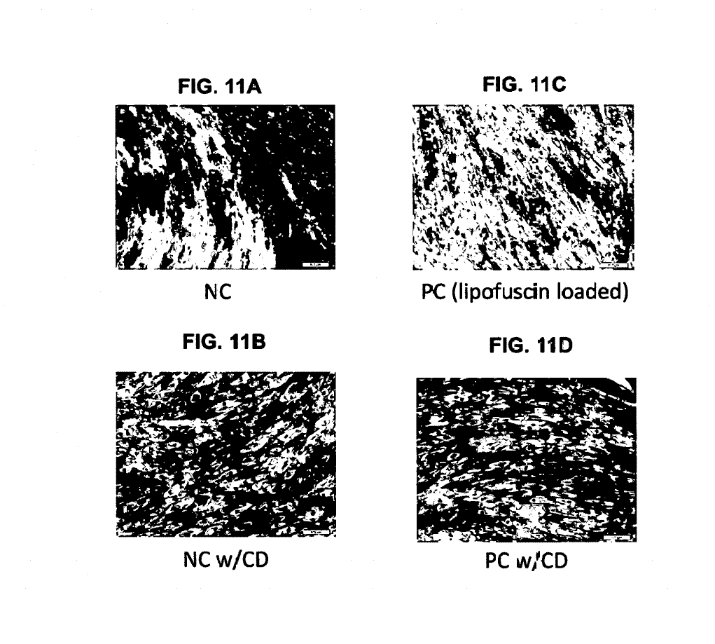

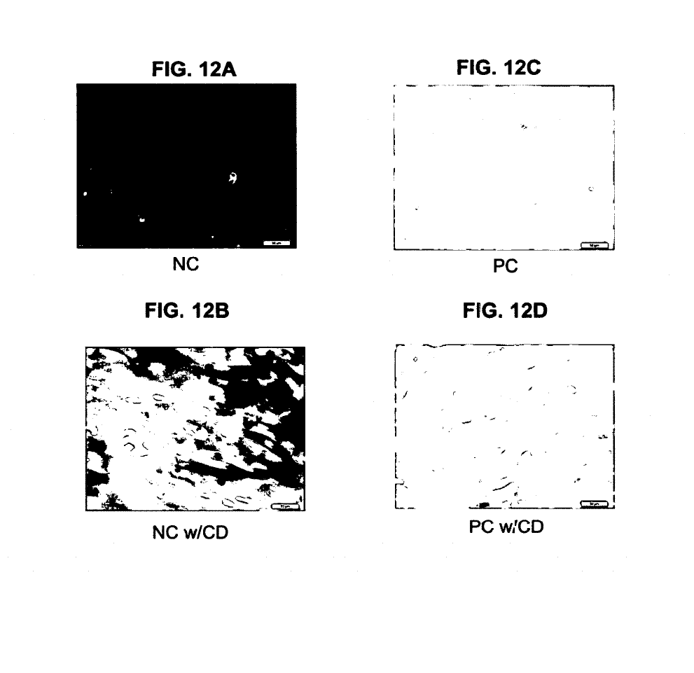

To further address the mechanism by which cyclodextrin treatment can decrease the concentration of lipofuscin in cells, filipin staining experiments were performed (FIG. 10A-D, FIG. 11A-D, and FIG. 12A-D). Filipin stain can be used to visualize the cholesterol present in a cell population. FIG. 10A and FIG. 10C provide representative photomicrographs of two different cell populations before confluency. Four days after the time the micrographs in FIG. 10A and FIG. 10C were produced, a filipin stain was applied to both cell populations (FIG. 10B and FIG. 10D), however, in the case of FIG. 10D, the cell population was treated with cyclodextrin for 3.5 hours before the filipin staining procedure. The cell population treated with cyclodextrin (FIG. 10D) showed a reduction in total cholesterol as measured by the filipin stain when compared to the untreated cell population (FIG. 10B). In order to measure the effects of long term cyclodextrin treatment, untreated healthy cells (NC), healthy cells treated with cyclodextrin (NC w/CD), untreated cells loaded with lipofuscin (PC), and cells loaded with lipofuscin and treated with cyclodextrin (PC w/CD) were filipin stained and imaged at 100× magnification after 4 days of maintenance (FIG. 11A-D) and were filipin stained and imaged at 200× magnification after 9 days of cell maintenance (FIG. 12A-D). The representative photomicrographs of FIG. 11A-D further demonstrated the ability of cyclodextrin treatment to cause a reduction of the cholesterol content of cells as visualized by the filipin stain, however, the representative photomicrographs of FIG. 12A-D demonstrated that long term treatment with cyclodextrin may increase the overall cellular levels of cholesterol as visualized by filipin stain. The results of FIG. 11A-D and FIG. 12-D indicate that the cyclodextrin treatment resulted in increased cholesterol levels in cells over time. Thus, it is believed that cyclodextrin treatment resulted in cholesterol efflux causing replacement of “old” (oxidized) cholesterol, and over time the cholesterol level in the cells was increased.

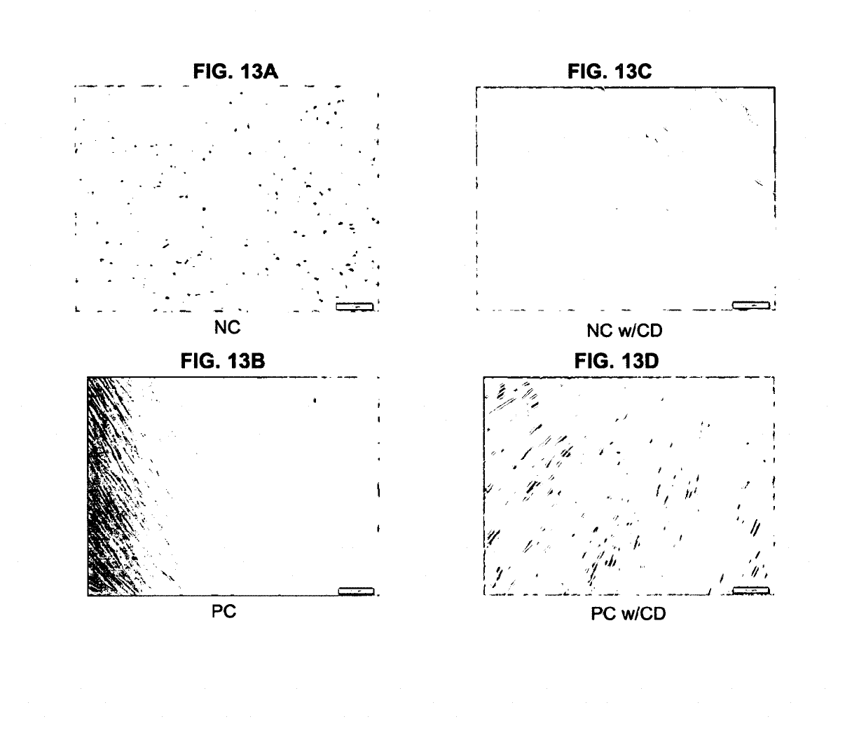

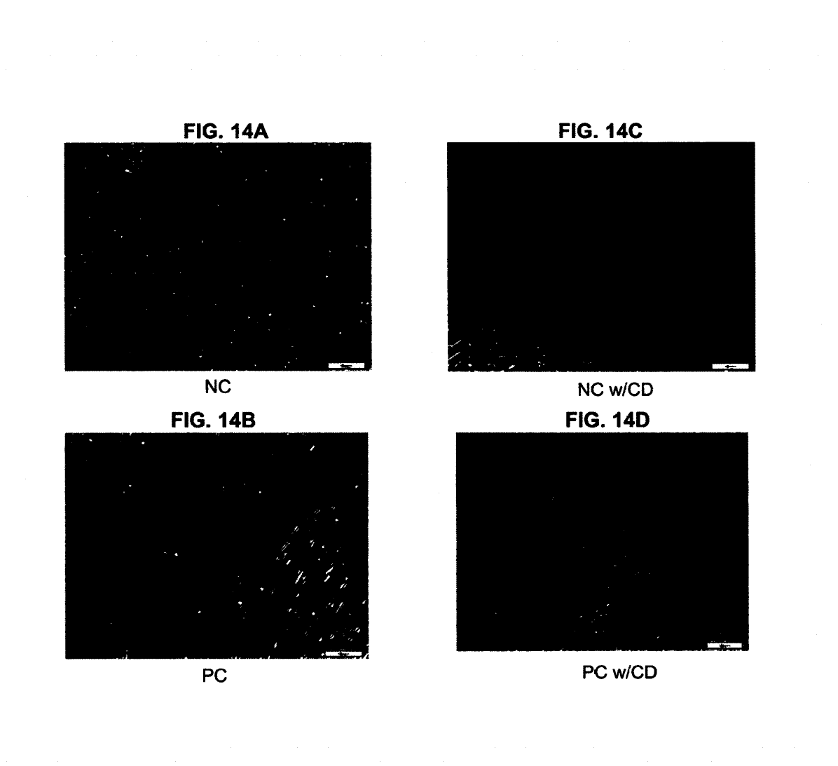

To further elucidate the mechanism by which cyclodextrin can decrease the concentration of lipofuscin in cells, an assay in which the lysomotropic agent MSDH was administered to cells in order to measure the effect of cyclodextrin administration on cellular health post-MSDH treatment was performed (FIG. 13A-D and FIG. 14A-D). FIG. 13A-D provides representative photomicrographs of NC, PC, NC w/CD, and PC w/CD cell populations after 42 hours of exposure to 45 μM of MSDH. NC cells were killed by exposure to 45 μM of MSDH (FIG. 13A), while PC (FIG. 13B), NC w/CD (FIG. 13C), and PC w/CD (FIG. 13D) cell populations were able to survive treatment with 45 μM of MSDH. FIG. 14A-D provides representative photomicrographs of NC (FIG. 14A), PC (FIG. 14B), NC w/CD (FIG. 14C), and PC w/CD (FIG. 14D) cell populations, respectively, after the populations were exposed to an increased level of MSDH for an increased duration of time (55 μM for 42 hours). Following said exposure to increased MSDH for an increased duration of time, the NC cell population experienced complete death (FIG. 14A), the PC cell population experienced moderate death (FIG. 14B), the NC w/CD cell population experienced moderate death (FIG. 14C), and the PC w/CD cell population experienced no death (FIG. 14D). The results of FIG. 14A-D were indicative of different degrees of lysosomal membrane cholesterol composition change pending cyclodextrin treatment and lipofuscin levels, with the aged lipofuscin loaded cells treated with cyclodextrin being the most resistant to MSDH treatment (FIG. 14D).

Taken together, the results provided in FIG. 9, FIG. 10A-D, FIG. 11A-D, FIG. 12A-D, FIG. 13A-D, and FIG. 14A-D indicated that HPβCD could function by modulating the total cellular cholesterol content as well as the cholesterol composition of the lysosomal membrane. These data additionally indicated that HPβCD could manipulate membrane compositions at both the plasma membrane and organelle level, and that this change of membrane composition may be responsible for or may be directly associated with the removal of lipofuscin.

Each of the following references is incorporated by reference herein in its entirety for all purposes:

Allaire, J., et al., Lipofuscin accumulation in the vastus lateralis muscle in patients with chronic obstructive pulmonary disease. Muscle & nerve 2002, 25, (3), 383-389.

Brunk, U. T. & Terman, A, Lipofuscin: mechanisms of age-related accumulation and influence on cell function. Free Radical Biology and Medicine 2002,33, (5), 611-619.

Ivy, G., et al., Inhibitors of lysosomal enzymes: accumulation of lipofuscin-like dense bodies in the brain. Science 1984, 226, (4677), 985-987.

Ju, S. J., et al., Use of extractable lipofuscin for age determination of blue crab Callinectes sapidus. Marine Ecology Progress Series 1999, 185, (1), 171-179.

Nociari, M. M., et al., Beta cyclodextrins bind, stabilize, and remove lipofuscin bisretinoids from retinal pigment epithelium. Proceedings of the National Academy of Sciences 2014,111, (14), EI402-E140S.

Puckett, B. J., et al., Validation and application of lipofuscin-based age determination for Chesapeake Bay blue crabs Callinectes sapidus. Transactions of the American Fisheries Society 2008,137, (6), 1637-1649.

Rosenbaum, A. I. & Maxfield, F. R, Niemann-Pick type C disease: molecular mechanisms and potential therapeutic approaches. Journal of neurochemistry 2011, 116, (5), 789-795.

Rosenbaum, A. I., et al., Endocytosis of betacyclodextrins is responsible for cholesterol reduction in Niemann-Pick type C mutant cells, Proceedings of the National Academy of Sciences 2010, 107, (12), 5477-5482.

SITTE, N.; et al., Proteasome inhibition by lipofuscin/ceroid during postmitotic aging of fibroblasts. The FASEB Journal 2000, 14, (11), 1490-1498.

Song, W., et al., 2-Hydroxypropyl-B-cyclodextrin Promotes Transcription Factor EB-mediated Activation of Autophagy IMPLICATIONS FOR THERAPY. Journal of Biological Chemistry 2014, 289, (14), 10211-10222.

Terman, A, Garbage catastrophe theory of aging: imperfect removal of oxidative damage? Redox Report 2001, 6, (1), 15-26.

Terman, A; Brunk, U. T., Ceroid lipofuscin formation in cultured human fibroblasts: the role of oxidative stress and lysosomal proteolysis. Mechanisms of ageing and development 1998, 104, (3), 277-291.

TERMAN, A; BRUNK, U. T., Lipofuscin: mechanisms of formation and increase with age. Apmis 1998, 106, (1-6), 265-276.

Terman, A., et al., The lysosomal-mitochondrial axis theory of postmitotic aging and cell death. Chemica-biological interactions 2006,163, (1),29-37.

Terman, A., et al., Mitochondrial turnover and aging of long-lived postmitotic cells: the mitochondrial-lysosomal axis theory of aging. Antioxidants & redox signaling 2010, 12, (4), 503-535.

U.S. Pat. No. 4,877,778 Injecting water soluble 2-hydroxypropyl-b-cyclodextrin

U.S. Pat. No. 5,206,224 Lowering level of male hormone by administering etherified cyclodextrin

U.S. Pat. No. 5,441,944 Substituted cyclodextrin sulfates and their uses as growth modulating agents

U.S. Pat. No. 5,637,575 Orally administering liquid composition comprising water soluble sulfated cyclodextrin derivative arid nontoxic carrier

WO2014022841 Cyclodextrin for the treatment of lysosomal storage diseases

Wu Y & Ke J. L., Structures and biogenetic analysis of lipofuscin bis-retinoids, Journal of Zhejiang University-SCIENCE B (Biomedicine & Biotechnology) 14(9):763-773 (2013).

What is claimed is:

1. A method of treating or delaying the onset of an age-associated phenotype in a cell, comprising contacting said cell with an effective amount of at least one cyclodextrin.

2. A method of treating or delaying the onset of an age-associated phenotype in a cell, comprising contacting said cell with an effective amount of at least one cyclodextrin comprising 2-hydroxypropyl-β-cyclodextrin, or a derivative thereof, a β-cyclodextrin or a derivative thereof, an α-cyclodextrin or a derivative thereof, and/or a γ-cyclodextrin or a derivative thereof.

3. A method of treating or delaying the onset of an age-associated phenotype in a cell, comprising contacting said cell with an effective amount of 2-hydroxypropyl-β-cyclodextrin.

4. The method of any one of claims 1-3, which is conducted in vitro.

5. The method of claim 4, further comprising implanting said cell into a subject.

6. The method of claim 5, wherein said cell is derived from said subject.

7. A method of treating or delaying the onset of geriatric aging of the human or animal body, tissue, or organ comprising administering a cyclodextrin compound to a subject.

8. An in vivo method of treating or delaying the onset of an age-associated phenotype in a cell, comprising administering to a subject an effective amount of at least one cyclodextrin comprising: 2-hydroxypropyl-β-cyclodextrin, or a derivative thereof, a β-cyclodextrin or a derivative thereof, an α-cyclodextrin or a derivative thereof, or a γ-cyclodextrin or a derivative thereof.

9. An in vivo attenuating or delaying the onset of an age-associated phenotype in a cell, comprising administering to a subject an effective amount of 2-hydroxypropyl-β-cyclodextrin.

10. The method of any one of claims 7-9, wherein the treatment is administered topically or parenterally.

11. The method of any one of claims 7-9, wherein the treatment is administered intravenously, orally, nasally, via the cerebrospinal fluid, via implant, transmucosally, transdermally, intramuscularly, intraocularly, parenterally, topically or subcutaneously.

12. The method of any one of claims 5-11, which delays the onset of a disease associated with aging.

13. The method of any one of claims 5-12, wherein said treatment is initiated when the age of said subject is less than 40, less than 35, less than 30, less than 25, or less than 20 years.

14. The method of any one of claims 5-13, wherein said subject exhibits pre-hypertension, metabolic syndrome, and/or fatty streaks.

15. The method of any one of claims 5-14, which is for the prevention, treatment, or to delay the onset of a disease or condition related to aging.

16. The method of any one of claims 5-14, which is for the prevention, treatment, or to delay the onset of a disease or disorder selected from: cardiovascular disease, cerebrovascular diseases, neurodegenerative diseases, eye diseases, inflammatory diseases, age-related diseases and conditions, or lipofuscin-associated disorders.

17. The method of any one of claims 5-14, which is for the prevention, treatment, or to delay the onset of cardiovascular and cerebrovascular diseases and conditions such as arteriosclerosis, coronary heart disease, arrhythmia, heart failure, hypertension, orthostatic hypotension, myocardial infarction, angina pectoris, heart failure, stroke, heart disease, and congenital heart disease; cancers such as endometrial cancer, melanomas, colon cancer, uterine cancer, ovarian cancer, pancreatic cancer, cervical cancer, bladder cancer, brain cancer, breast cancer, lung cancer, and prostate cancer; neurodegenerative diseases such as Parkinson's disease and Alzheimer's disease; celiac disease; atherosclerosis; dyslipidemia; arthritis; rheumatoid arthritis; age-related eye diseases such as macular degeneration, Stargardt disease, cataracts, diabetic retinopathy, and glaucoma; hearing loss; renal artery disease or stenosis; peripheral vascular disease; chronic obstructive pulmonary disease (“COPD”); chronic renal diseases with renal failure; ulcers; osteopetrosis; progeria; and type-2 diabetes.

18. The method of any one of claims 5-14, which is for the prevention or treatment of liver spots.

19. The method of claim 18, wherein said treatment is administered topically.

20. The method of any one of the foregoing claims, wherein the cell is a human cells or a non-human animal cell.

21. The method of any of the foregoing claims, which attenuates lysosomal destabilization and/or lysosomal permeability in the cell.

22. The method of any of the foregoing claims, which decreases the concentration of 7-ketocholesterol in the cell.

23. The method of any of the foregoing claims, wherein the treatment modulates the total cellular cholesterol content or the cholesterol composition of the lysosomal membrane.

24. The method of any of the foregoing claims, which reduces lipofuscin in said cells.

25. The method of any of the foregoing claims, which attenuates lysosomal destabilization and/or lysosomal permeability caused by 7-ketocholesterol.

26. The method of any of the foregoing claims, wherein said cyclodextrin is of the structure: 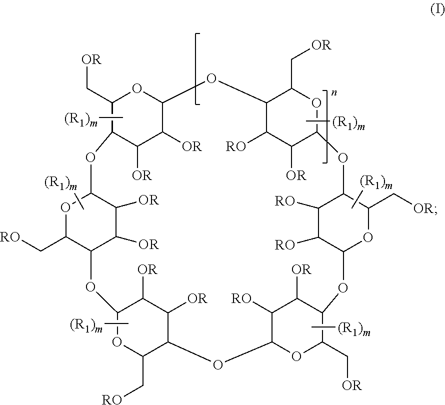
or a pharmaceutically acceptable salt, ester, solvate, or hydrate thereof.

27. The method of claim 26, wherein, each R is independently H, alkyl, alkenyl, alkynyl, cycloalkyl, heterocycloalkyl, aryl, or heteroaryl, each of which is optionally substituted; or —C(O)OR^{B}, —OC(O)R^{B}, —C(O)R^{B}, or —C(O)NR^{A}R^{B};

28. The method of any one of claims 26-27, wherein each R₁ is independently H, alkyl, alkenyl, alkynyl, cycloalkyl, aryl, heteroaryl, halogen, hydroxy, amino, —CN, —CF₃, —N₃, —NO₂, —OR^{B}, —SR ^{B}, —SOR^{B}, —SO₂R^{B}, —N(R^{B})S(O₂), —R^{B}, —N(R^{B})S(O₂)NR^{A}R^{B}, —NR^{A}R^{B}, —C(O)OR^{B}, —OC(O)R^{B}, —C(O)R^{B}, —C(O)NR^{A}R^{B}, or —N(R^{B})C(O)R^{B}; each of which is optionally substituted;

29. The method of any one of claims 26-28, wherein each R^{A} is independently hydrogen, alkyl, alkenyl, alkynyl, cycloalkyl, heterocycloalkyl, aryl, or heteroaryl, each of which is optionally substituted;

30. The method of any one of claims 26-29, wherein each R^{B} is independently hydrogen, alkyl, alkenyl, alkynyl, cycloalkyl, heterocycloalkyl, aryl, or heteroaryl, each of which is optionally substituted;

31. The method of any one of claims 26-30, wherein n is 1, 2, 3, 4, 5, 6, 7, 8, 9, or 10;

32. The method of any one of claims 26-31, wherein each m is independently 0, 1, 2, 3, 4, or 5.

33. The method according to any one of the foregoing claims, wherein said cyclodextrin is the sole active ingredient utilized in said method.

34. .A composition comprising at least one cyclodextrin or derivative thereof which is suitable for use in a method according to any one of the foregoing claims, which optionally further comprises a carrier or excipient.

35. A composition for treating a disease associated with aging, comprising a cyclodextrin and optionally further comprises a pharmaceutically acceptable carrier or excipient.

36. The composition of claim 36, which comprises a cyclodextrin as recited in any one of claims 26-32.

37. The composition of claim 35 or 3630 or 31, which is for the treatment of a disease related to aging.

38. The composition of claim 35 or 36, which is for the treatment of a disease or disorder selected from: cardiovascular disease, cerebrovascular diseases, neurodegenerative diseases, eye diseases, inflammatory diseases, and age-related diseases and conditions.

39. The composition of claim 35 or 36, which is for the treatment of cardiovascular and cerebrovascular diseases and conditions such as arteriosclerosis, coronary heart disease, arrhythmia, heart failure, hypertension, orthostatic hypotension, myocardial infarction, angina pectoris, heart failure, stroke, heart disease, and congenital heart disease; cancers such as endometrial cancer, melanomas, colon cancer, uterine cancer, ovarian cancer, pancreatic cancer, cervical cancer, bladder cancer, brain cancer, breast cancer, lung cancer, and prostate cancer; neurodegenerative diseases such as Parkinson's disease and Alzheimer's disease; celiac disease; atherosclerosis; dyslipidemia; arthritis; rheumatoid arthritis; age-related eye diseases such as macular degeneration, Stargardt disease, cataracts, diabetic retinopathy, and glaucoma; hearing loss; renal artery disease or stenosis; peripheral vascular disease; chronic obstructive pulmonary disease (“COPD”); chronic renal diseases with renal failure; ulcers; osteopetrosis; progeria; and type-2 diabetes.

40. The composition of any one of claims of claims 35-39, which is suitable for administration topically, parenterally, or by injection.

41. The composition of any one of claims of claims 35-39, which is suitable for administration intravenously, orally, nasally, via the cerebrospinal fluid, via implant, transmucosally, transdermally, intramuscularly, intraocularly, parenterally, topically or subcutaneously.

42. The composition of any one of claims 35-41, wherein said cyclodextrin is the sole active ingredient in said composition.

43. A method of manufacturing a medicament, comprising admixing a cyclodextrin and a pharmaceutically acceptable carrier or excipient.

44. The method of claim 43, wherein said medicament is suitable for use in the method of any one of claims 1-33.

45. The method of claim 43 or 44, wherein said cyclodextrin comprises a cyclodextrin as recited in any one of claims 26-32.

46. A method of treating geriatric aging of the body, comprising parentally or orally administering to an aging patient a composition comprising 2-hydroxypropyl-β-cyclodextrin, or a derivative thereof, or β-cyclodextrin or a derivative thereof, or α-cyclodextrin or a derivative thereof, or γ-cyclodextrin or a derivative thereof solubilized in parentally acceptable solution.

47. A method of treating geriatric aging of the body, comprising parentally or orally administering a composition comprising 2-hydroxypropyl-β-cyclodextrin, or a derivative thereof, or β-cyclodextrin or a derivative thereof, or α-cyclodextrin or a derivative thereof, or y-cyclodextrin or a derivative thereof solubilized in parentally acceptable solution.

48. A method of treating geriatric aging of the body, comprising administering a composition comprising 2-hydroxypropyl-β-cyclodextrin, or a derivative thereof, or β-cyclodextrin or a derivative thereof, or α-cyclodextrin or a derivative thereof, or γ-cyclodextrin or a derivative thereof solubilized in parentally acceptable solution.

49. A method of reducing age-related non-bisretinoid lipofuscin in a patient having a buildup of same, said method comprising administering 2-hydroxypropyl-β-cyclodextrin, or a derivative thereof, or β-cyclodextrin or a derivative thereof, or α-cyclodextrin or a derivative thereof, or γ-cyclodextrin or a derivative thereof to a patient in an amount effective to reduce age-related non-bisretinoid lipofuscin levels in said patient.

50. A method of treating geriatric aging of the body, comprising parentally or orally administering to an aging patient a composition comprising 2-hydroxypropyl-β-cyclodextrin, or a derivative thereof solubilized in parentally acceptable solution.

51. A method of treating geriatric aging of the body, comprising parentally or orally administering a composition comprising 2-hydroxypropyl-β-cyclodextrin, or a derivative thereof, solubilized in parentally acceptable solution.

52. A method of treating geriatric aging of the body, comprising parentally or orally administering a composition comprising 2-hydroxypropyl-β-cyclodextrin, or a derivative thereof, solubilized in parentally acceptable solution.

53. A method of treating geriatric aging of the body, comprising administering a composition comprising 2-hydroxypropyl-β-cyclodextrin, or a derivative thereof, solubilized in parentally acceptable solution.

54. A method of reducing age-related non-bisretinoid lipofuscin in a patient having a buildup of same, said method comprising administering 2-hydroxypropyl-β-cyclodextrin, or a derivative thereof to a patient in an amount effective to reduce age-related non-bisretinoid lipofuscin levels in said patient.

55. The method of any one of claims 46-54, wherein said administered composition comprises a cyclodextrin as recited in any one of claims 26-32.
